(19) United States
(12) Patent Application Publication
(10) Pub. No.: US 2019/0008368 A1
(43) Pub. Date: Jan. 10, 2019

(54) SCANNING ENDOSCOPE

(71) Applicant: OLYMPUS CORPORATION, Tokyo (JP)

(72) Inventor: Katsunori KUMAI, Tokyo (JP)

(73) Assignee: OLYMPUS CORPORATION, Tokyo (JP)

(21) Appl. No.: 16/131,096

(22) Filed: Sep. 14, 2018

Related U.S. Application Data
(63) Continuation of application No. PCT/JP2016/083909, filed on Nov. 16, 2016.

(30) Foreign Application Priority Data
Mar. 17, 2016 (JP) 2016-053503

(51) Int. Cl. A61B 1/00; A61B 1/07; G02B 23/26

(52) U.S. Cl. CPC A61B 1/00172; A61B 1/00096; A61B 1/00167; A61B 1/00002; A61B 1/07; G02B 23/26; A61B 1/00126

(57) ABSTRACT

A scanning endoscope includes: an optical fiber for illumination including an incidence side to which illumination light enters and an irradiate side from which the illumination light is irradiated, and configured to irradiate an object from the irradiation side with the illumination light; a holder configured to hold the optical fiber; a plurality of drive elements arranged so as to make the optical fiber swing between the irradiation side and the holder, and configured to make the optical fiber swing according to a drive signal; and a cylinder made of metal, and securing the optical fiber and the plurality of drive elements, wherein the cylinder is electrically connected to a ground potential.

### CROSS REFERENCE TO RELATED APPLICATION

This application is a continuation application of PCT/JP2016/083909 filed on Nov. 16, 2016 and claims benefit of Japanese Application No. 2016-053503 filed in Japan on Mar. 17, 2016, the entire contents of which are incorporated herein by this reference.

### BACKGROUND OF INVENTION

### 1. Field of the Invention

The present invention relates to a scanning endoscope.

### 2. Description of the Related Art

Conventionally, there is a scanning endoscope apparatus which scans an object by illumination light and picks up an image. For example, Japanese Patent Application Laid-Open Publication No. 2015-80488 discloses a scanning endoscope apparatus including a scanning endoscope, which makes an optical fiber for illumination swing by an actuator, irradiates an object with illumination light, receives the reflected light of the object by an optical fiber for light reception, and picks up an image of the object.

In particular, a scanning endoscope in a medical field does not include an image pickup device such as a CCD in an insertion portion so that a diameter of the insertion portion can be narrowed and burdens on a patient can be reduced.

### SUMMARY OF THE INVENTION

A scanning endoscope as one aspect of the present invention includes: an optical fiber for illumination including an incidence side to which illumination light enters and an irradiate side from which the illumination light is irradiated, and configured to irradiate an object from the irradiation side with the illumination light; a holder configured to hold the optical fiber; a plurality of drive elements arranged so as to make the optical fiber swing between the irradiation side and the holder, and configured to make the optical fiber swing according to a drive signal; and a cylinder made of metal, and securing the optical fiber and the plurality of drive elements, wherein the cylinder is electrically connected to a ground potential.

### BRIEF DESCRIPTION OF THE DRAWINGS

FIG. 1 is a block diagram illustrating an outline configuration of a scanning endoscope apparatus, pertaining to an embodiment of the present invention;

FIG. 2 is a diagram illustrating a configuration inside a protective pipe of a scanning endoscope, pertaining to the embodiment of the present invention;

FIG. 3 is a sectional view of an actuator of an endoscope, pertaining to the embodiment of the present invention;

FIG. 4A is an explanatory diagram explaining a spiral scanning route of the scanning endoscope, pertaining to the embodiment of the present invention;

FIG. 4B is an explanatory diagram explaining the spiral scanning route of the scanning endoscope, pertaining to the embodiment of the present invention;

FIG. 5 is a perspective view illustrating a configuration of a holding portion of the scanning endoscope, pertaining to the embodiment of the present invention;

FIG. 6 is a diagram illustrating a configuration inside the protective pipe of the scanning endoscope, pertaining to a modification 1 of the embodiment of the present invention;

FIG. 7 is a diagram illustrating a configuration inside the protective pipe of the scanning endoscope, pertaining to a modification 2 of the embodiment of the present invention;

FIG. 8 is a diagram illustrating a configuration inside the protective pipe of the scanning endoscope, pertaining to a modification 3 of the embodiment of the present invention;

FIG. 9 is a diagram illustrating a configuration inside the protective pipe of the scanning endoscope, pertaining to a modification 4 of the embodiment of the present invention;

FIG. 10 is a diagram illustrating a configuration of an insertion portion of the scanning endoscope, pertaining to a modification 5 of the embodiment of the present invention; and

FIG. 11 is a perspective view illustrating a configuration of the holding portion of the scanning endoscope, pertaining to a modification 6 of the embodiment of the present invention.

### DETAILED DESCRIPTION OF THE PREFERRED EMBODIMENT

Hereinafter, the embodiment of the present invention will be described with reference to the drawings.

(Configuration)

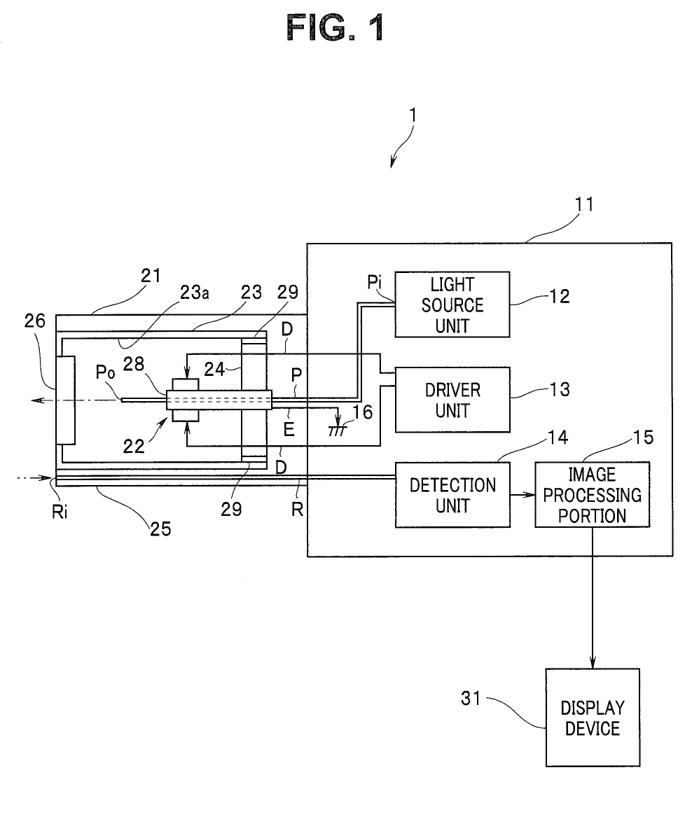

FIG. 1 is a block diagram illustrating an outline configuration of a scanning endoscope apparatus 1, pertaining to the embodiment of the present invention.

As illustrated in FIG. 1, the scanning endoscope apparatus 1 is configured including a scanning endoscope processor 11, a scanning endoscope 21, and a display device 31. The scanning endoscope processor 11 is connected to the scanning endoscope 21 and the display device 31.

The scanning endoscope processor 11 is configured including a light source unit 12, a driver unit 13, a detection unit 14, an image processing portion 15, and a patient GND 16.

The light source unit 12 is configured so as to be able to generate red, green and blue laser beams. The light source unit 12 is connected to an optical fiber P for illumination. The light source unit 12 successively and repeatedly generates laser beams from respective red, green and blue laser beam sources not illustrated, and makes the laser beams be incident on an incidence side end portion Pi of the optical fiber P for illumination, through a multiplexer not illustrated.

The optical fiber P for illumination is configured so as to be able to irradiate an object from an irradiation side end portion Po with illumination light made incident from an incidence side end portion Pi. Of the optical fiber P for illumination, the incidence side end portion Pi is connected to the light source unit 12, and the irradiation side end portion Po is arranged inside the scanning endoscope 21. The optical fiber P for illumination guides the laser beam made incident from the light source unit 12 to the irradiation side end portion Po of the optical fiber P for illumination inside the scanning endoscope 21, and irradiates the object from the irradiation side end portion Po with the laser beam as the illumination light.

The driver unit 13 is a circuit configured to drive an actuator 22 inside the scanning endoscope 21, and make the irradiation side end portion Po of the optical fiber P for illumination swing. The driver unit 13 is connected to the actuator 22 by a drive signal line D. The driver unit 13 generates drive signals AX and AY by a signal generator not illustrated, and outputs the drive signals AX and AY to the actuator 22 through the drive signal line D.

The drive signal AX is defined by an equation (1) below, for example. In the equation (1) below, X(t) denotes a signal level of the drive signal AX at time t, Mx denotes an amplitude value independent of the time t, and Gx(t) denotes a predetermined function which modulates a sine wave sin(2Πft).

X(t)=Mx×Gx(t)×sin(2Πft) (1)

The drive signal AY is defined by an equation (2) below, for example. In the equation (2) below, Y(t) denotes a signal level of the drive signal AY at the time t, My denotes an amplitude value independent of the time t, Gy(t) denotes a predetermined function which modulates a sine wave sin(2Πft+φ), and φ denotes a phase of the drive signal AX.

Y(t)=My×Gy(t)×sin(2Πft+φ) (2)

The detection unit 14 is a circuit configured to detect reflected light of the object. The detection unit 14 is configured including a photoelectric conversion element not illustrated. The detection unit 14 is connected to an optical fiber R for light reception of the scanning endoscope 21, and to the image processing portion 15. When the object is irradiated with the illumination light from the irradiation side end portion Po of the optical fiber P for illumination, a light receiving portion Ri receives the reflected light of the object. The detection unit 14 converts the reflected light of the object inputted from the light receiving portion Ri through the optical fiber R for light reception to an image pickup signal, and outputs the signal to the image processing portion 15.

The image processing portion 15 is a circuit configured to process an image. The image processing portion 15 is connected to the display device 31. Based on the image pickup signal inputted from the detection unit 14, the image processing portion 15 refers to a mapping table not illustrated, performs various kinds of image correction processing, generates an output image for each frame, and outputs the image to the display device 31.

The patient GND 16 is a ground terminal of a patient circuit. The patient GND 16 is separated from a device side circuit connected to a commercial power source. The patient GND 16 is connected to the ground potential. The ground potential of the patient GND 16 is a reference potential of the drive signals AX and AY. The patient GND 16 is connected to a ferrule 28.

The display device 31 is configured so as to be able to display the output image outputted from the scanning endoscope processor 11.

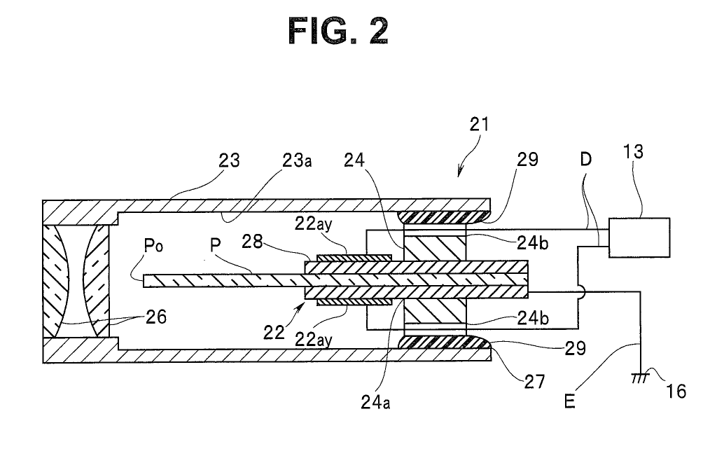

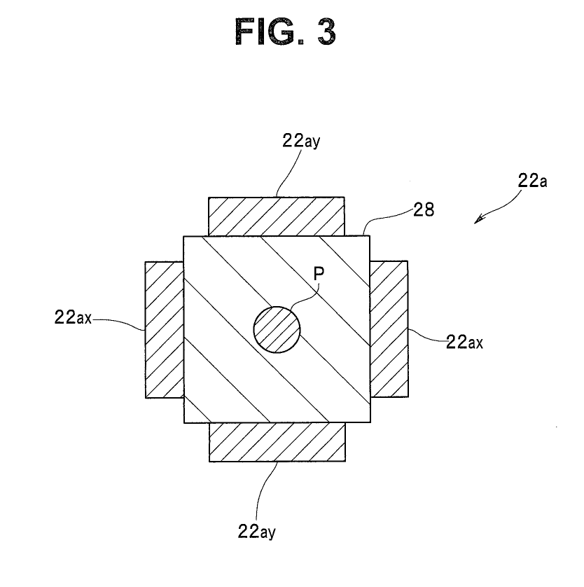

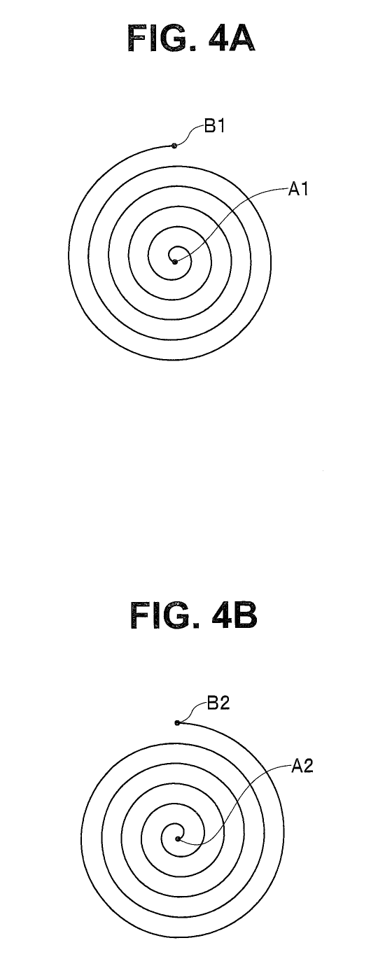

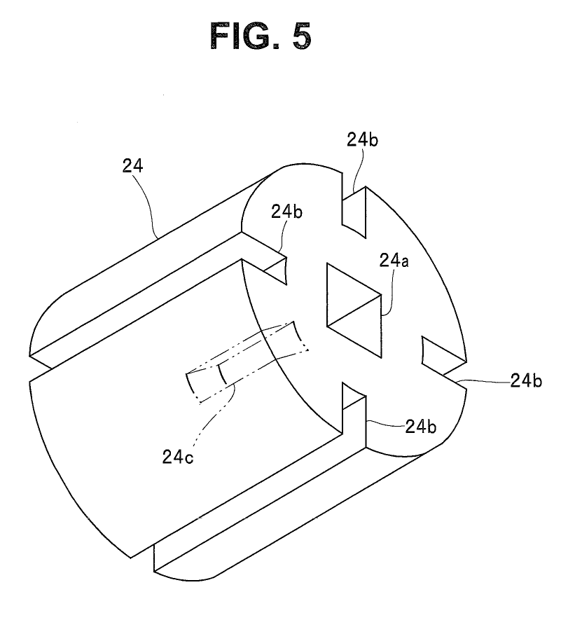

FIG. 2 is a diagram illustrating a configuration inside a protective pipe 23 of the scanning endoscope 21, pertaining to the embodiment of the present invention. FIG. 3 is a sectional view of the actuator 22 of the scanning endoscope 21, pertaining to the embodiment of the present invention. FIG. 4A and FIG. 4B are explanatory diagrams explaining a spiral scanning route of the scanning endoscope 21, pertaining to the embodiment of the present invention. FIG. 5 is a perspective view illustrating a configuration of a holding portion 24 (holder) of the scanning endoscope 21, pertaining to the embodiment of the present invention.

The scanning endoscope 21 is configured so as to be able to insert an insertion portion 25 (FIG. 1) to the object, irradiate the object with the illumination light emitted by the light source unit 12, and pick up the image of the object. The scanning endoscope 21 is configured including the insertion portion 25, the actuator 22, the holding portion 24 and a lens 26.

The insertion portion 25 is configured so as to be inserted into the object. The insertion portion 25 is formed in an elongated shape and configured including the protective pipe 23 which is a cylinder body (cylinder) at a distal end.

As illustrated in FIG. 2, the protective pipe 23 is configured with metal as a material. The protective pipe 23 is formed in a tubular shape. More specifically, the protective pipe 23 is a cylindrical shape. Inside the protective pipe 23, the optical fiber P for illumination and the actuator 22 are arranged and are fixed to the protective pipe 23 by the holding portion 24.

The optical fiber P for illumination is interpolated from a base end 27 of the protective pipe 23 such that the irradiation side end portion Po is arranged inside the protective pipe 23. The optical fiber P for illumination is provided with the ferrule 28 on a portion of an outer periphery.

The ferrule 28 is configured with metal as a material. To the ferrule 28, a ground line E is connected, and the ferrule 28 is connected to the patient GND 16 inside the scanning endoscope processor 11 through the ground line E. The ferrule 28 is formed in a square pole shape but, without being limited to the square pole shape, may be a polygonal prism shape or a columnar shape, for example.

The actuator 22 is arranged between the irradiation side end portion Po of the optical fiber P for illumination and the holding portion 24 holding the optical fiber P for illumination, and configured so as to be able to make the optical fiber P for illumination swing. The actuator 22 includes piezoelectric elements 22a which are a plurality of drive elements that make the optical fiber P for illumination swing according to the drive signals AX and AY.

The piezoelectric elements 22a are arranged so as to surround the outer periphery of the ferrule 28. In FIG. 3, two piezoelectric elements 22ax for an X axis that make the optical fiber P for illumination swing in an X axis direction and two piezoelectric elements 22ay for a Y axis that make the optical fiber P for illumination swing in a Y axis direction are arranged so as to surround the outer periphery of the ferrule 28.

Each of the piezoelectric elements 22a is formed in a planar shape. An electrode arranged on one surface of each piezoelectric element 22a is attached to the drive signal line D, and connected to the driver unit 13 through the drive signal line D. An electrode arranged on the other surface of each piezoelectric element 22a is attached to the ferrule 28, and connected to the patient GND 16 through the ferrule 28.

When the driver unit 13 outputs the drive signals AX and AY while increasing the signal level, the optical fiber P for illumination is made to swing by the actuator 22, and an irradiation position of a laser beam of the optical fiber P for illumination is moved along a spiral scanning route gradually becoming more distant from a center, as illustrated from A1 to B1 in FIG. 4A. Thereafter, when the driver unit 13 outputs the drive signals AX and AY while reducing the signal level, the irradiation position of the laser beam of the optical fiber P for illumination is moved along a spiral scanning route gradually approaching the center, as illustrated from B2 to A2 in FIG. 4B. Thus, the object is irradiated spirally with respective red, green and blue laser beams, and the reflected light of the object is received by the light receiving portion Ri (FIG. 1).

The holding portion 24 is configured so as to be able to hold the optical fiber P for illumination. The holding portion 24 is configured with metal as a material. The holding portion 24 is formed in a short pillar shape so as to be internally fitted to the base end 27 of the protective pipe 23. More specifically, the holding portion 24 is formed in a short columnar shape. The holding portion 24 is fixed to the protective pipe 23 with a conductive adhesive material 29 so as to be electrically connected with the protective pipe 23.

That is, the holding portion 24 is internally fitted to the cylinder body such that at least a portion of the outer periphery of the holding portion 24 and at least a portion of an inner wall 23a of the protective pipe 23 are arranged to overlap each other, and is electrically connected with the protective pipe 23.

The holding portion 24 includes an optical fiber attaching portion 24a for attaching the optical fiber P for illumination and a signal line arranging portion 24b configured to arrange the drive signal line D configured to transmit the drive signals AX and AY.

The optical fiber attaching portion 24a is configured of a center fitting hole passing through the holding portion 24 in an axial direction. In FIG. 5, the optical fiber attaching portion 24a is configured of a quadrangular fitting hole. To the optical fiber attaching portion 24a, the ferrule 28 provided on the outer periphery of the optical fiber P for illumination is fitted. Since the ferrule 28 is fitted to the optical fiber attaching portion 24a, the holding portion 24 holds the optical fiber P for illumination together with the ferrule 28. Thus, the holding portion 24 is electrically connected with the ferrule 28 connected to the patient GND 16. Since the protective pipe 23 and the holding portion 24 are also electrically connected, the protective pipe 23 is electrically connected to the patient GND 16 which is the ground potential.

The signal line arranging portion 24b is configured of a through groove provided on the outer periphery of the holding portion 24 in the axial direction. In FIG. 5, the signal line arranging portion 24b is configured of four through grooves. At the signal line arranging portion 24b, the drive signal line D connected to the driver unit 13 is arranged. The drive signal line D is covered for insulation with rubber of vinyl or the like. Therefore, the signal line arranging portion 24b of the holding portion 24 is insulated from the drive signal line D.

The lens 26 is provided on a distal end of the protective pipe 23, and is configured so as to be able to irradiate the object with the illumination light irradiated from the irradiation side end portion Po of the optical fiber P for illumination.

Thus, the protective pipe 23 is electrically connected to the ground potential of the patient GND 16 through the holding portion 24. Even in a case where the protective pipe 23 receives an impact from outside, the actuator 22 is brought into contact with the inner wall 23a of the cylinder body due to bending of the protective pipe 23 or breakage of the holding portion 24, for example, and the actuator 22 and the protective pipe 23 are conducted, a current flowing from the actuator 22 into the protective pipe 23 flows into the patient GND 16 through the holding portion 24, the ferrule 28 and the ground line E. In other words, the protective pipe 23 is electrically connected to the ground potential, and even in the case of being conducted with the actuator 22, the ground potential is maintained. Therefore, in the scanning endoscope 21, generation of an abnormal voltage in the protective pipe 23 is suppressed.

In the scanning endoscope 21, the ground line E is connected to the ferrule 28. Therefore, the ground line E is not arranged inside the protective pipe 23, and a diameter of the protective pipe 23 can be narrowed.

According to the above-described embodiment, in the scanning endoscope 21, the protective pipe 23 at the distal end of the insertion portion 25 is formed of metal, and even in the case where the actuator 22 is brought into contact with the inner wall 23a of the protective pipe 23 by an impact from the outside, the generation of an abnormal voltage in the protective pipe 23 is suppressed.

### Modification 1 of the Embodiment

In the embodiment, the holding portion 24 is fixed to the base end 27 of the protective pipe 23 with the conductive adhesive material 29. However, the holding portion 24 may be fixed without using the conductive adhesive material 29.

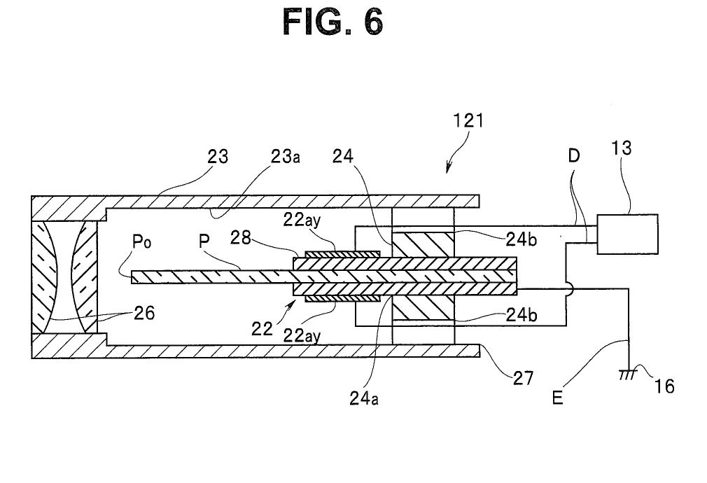

FIG. 6 is a diagram illustrating a configuration inside the protective pipe 23 of a scanning endoscope 121, pertaining to the modification 1 of the embodiment of the present invention.

As illustrated in FIG. 6, the holding portion 24 of the scanning endoscope 121 is internally fitted and fixed to the base end 27 of the protective pipe 23. More specifically, the holding portion 24 is fixed by being press-fitted to the base end 27 of the protective pipe 23.

Thus, in the scanning endoscope 121, the holding portion 24 can be fixed to the protective pipe 23 without using the conductive adhesive material 29, and a production process can be simplified.

### Modification 2 of the Embodiment

In the embodiment, the ground line E is attached to the ferrule 28. However, the ground line E may be attached to the protective pipe 23.

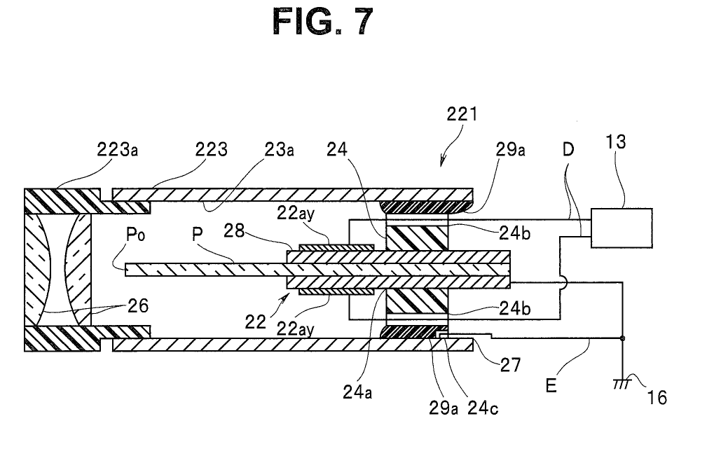

FIG. 7 is a diagram illustrating a configuration inside a protective pipe 223 of a scanning endoscope 221, pertaining to the modification 2 of the embodiment of the present invention.

As illustrated in FIG. 7, to the protective pipe 223, the ground line E is attached, and the protective pipe 223 is connected to the patient GND 16 through the ground line E.

In the modification 2, the holding portion 24 may be configured with a resin such as plastic as a material. The holding portion 24 includes a ground line arranging portion 24c (two-dot chain lines in FIG. 5). At the ground line arranging portion 24c, the ground line E configured to transmit the ground potential is arranged, and the ground line E is fixed in a state where the ground line E and the protective pipe 223 are electrically connected. The holding portion 24 is internally fitted to the protective pipe 223, and is fixed with an adhesive material 29a. The adhesive material 29a may not be conductive.

A lens frame 223a is configured with a resin such as plastic as a material. The lens frame 223a is formed in a short tubular shape, and is configured so as to be able to hold the lens 26 on an inner side. The lens frame 223a is fixed to the protective pipe 223 by being internally fitted to the distal end of the protective pipe 223.

Thus, in the scanning endoscope 221, the protective pipe 223 is formed of metal, and even in the case where the protective pipe 223 receives an impact from the outside and the actuator 22 and the protective pipe 223 are conducted, the generation of an abnormal voltage in the protective pipe 223 is suppressed.

In addition, in the scanning endoscope 221, the distal end of the protective pipe 223 is configured of the resin lens frame 223a, and an insulation property to the object and an operator at the distal end of the insertion portion 25 is improved.

### Modification 3 of the Embodiment

In the embodiment, the ground line E is connected to the ferrule 28. However, the outer periphery of the protective pipe 23 may be wound with a metal sheet 223b and the ground line E may be connected to the metal sheet 223b.

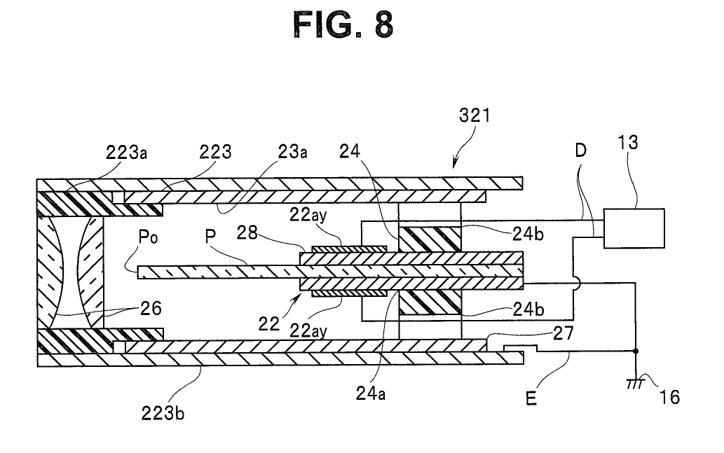

FIG. 8 is a diagram illustrating a configuration inside the protective pipe 223 of a scanning endoscope 321, pertaining to the modification 3 of the embodiment of the present invention.

As illustrated in FIG. 8, the outer periphery of the protective pipe 223 of the scanning endoscope 321 is wound with the metal sheet 223b.

The metal sheet 223b is configured of a conductive material such as a metal tape, metal mesh or metal foil of copper or aluminum or the like. The metal sheet 223b is wound around the outer periphery of the lens frame 223a and the protective pipe 223. The metal sheet 223b is connected to the ground line E and is connected to the patient GND 16 through the ground line E.

That is, the protective pipe 223 is wound with the metal sheet 223b, which is the conductive material, on at least a portion of the outer periphery, and the metal sheet 223b is connected to the ground potential.

Thus, in the scanning endoscope 321, the protective pipe 223 is formed of metal, and even in the case where the protective pipe 223 receives an impact from the outside and the actuator 22 and the protective pipe 223 are conducted, the generation of an abnormal voltage in the protective pipe 223 is suppressed.

In the scanning endoscope 321, an area of a mutual contact part of the protective pipe 223 and the metal sheet 223b is large, and electric resistance of the contact part of the protective pipe 223 and the metal sheet 223b is small. Further, in the scanning endoscope 321, by extending the metal sheet 223b further from the base end 27 of the protective pipe 223, for example, an area of a connection part with the ground line E can be widened, the electric resistance of the connection part of the metal sheet 223b and the ground line E can be reduced, and attachment of the ground line E can be facilitated.

### Modification 4 of the Embodiment

In the embodiment, the holding portion 24 is internally fitted to the protective pipe 23. However, the holding portion 24 may be integrated with the protective pipe 23.

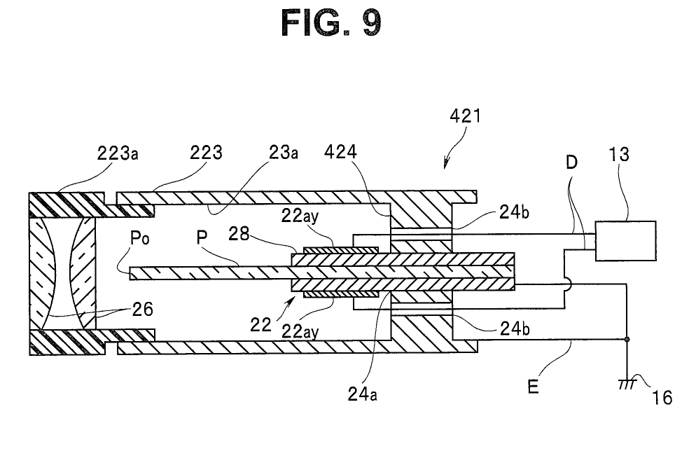

FIG. 9 is a diagram illustrating a configuration inside the protective pipe 223 of a scanning endoscope 421, pertaining to the modification 4 of the embodiment of the present invention.

As illustrated in FIG. 9, the protective pipe 223 of the scanning endoscope 421 is formed of metal integrated with a holding portion 424.

Thus, in the scanning endoscope 421, the protective pipe 223 is formed of metal, and even in the case where the protective pipe 223 receives an impact from the outside and the actuator 22 and the protective pipe 223 are conducted, the generation of an abnormal voltage in the protective pipe 223 is suppressed.

### Modification 5 of the Embodiment

In the embodiment, the protective pipe 23 is connected to the patient GND 16 through the ground line E. However, the protective pipe 23 may be connected to the patient GND 16 through a conductive portion Tc inside a casing tube T.

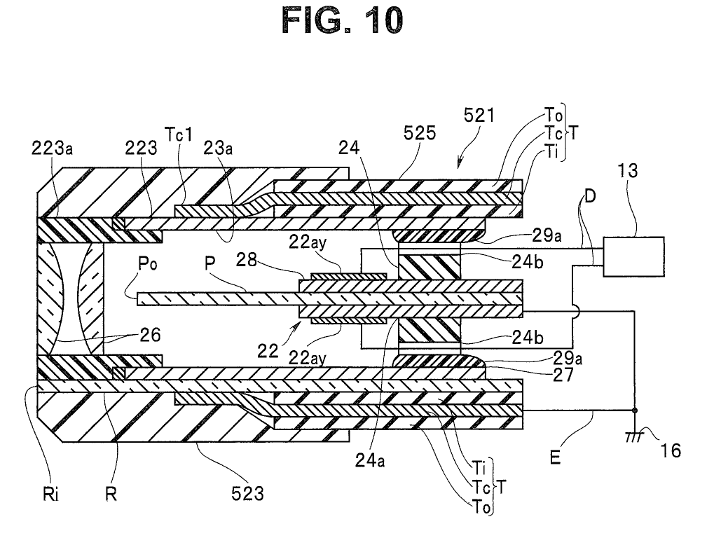

FIG. 10 is a diagram illustrating a configuration of an insertion portion 525 of a scanning endoscope 521, pertaining to the modification 5 of the embodiment of the present invention.

In the modification 5 of the embodiment, in the scanning endoscope 521, the optical fiber R for light reception including the light receiving portion Ri is arranged on the outer periphery of the protective pipe 223, the casing tube T is arranged further on an outer side of the optical fiber R for light reception, and a cap 523 is arranged so as to cover the distal end of the casing tube T from the distal end of the insertion portion 25.

In the casing tube T, an inner layer Ti and an outer layer To are configured of an insulating member such as rubber or vinyl, and the conductive portion Tc configured of a metal mesh tube, for example, is provided between the inner layer Ti and the outer layer To. The casing tube T is attached to the protective pipe 223. On a distal end side of the casing tube T, a conductive portion Tc1 is extended, and connected to the outer periphery of the protective pipe 223. On a base end side of the casing tube T, the conductive portion Tc is connected to the patient GND 16 through the ground line E. That is, the casing tube T is attached to the protective pipe 223, the casing tube T is configured including the conductive portion Tc, and the protective pipe 223 is connected to the patient GND 16 through the conductive portion Tc and the ground line E.

The cap 523 is configured with a resin such as plastic as a material. The cap 523 is formed in a short tubular shape, and is configured so as to be fitted to the outer periphery of the protective pipe 223.

Thus, in the scanning endoscope 521, the protective pipe 223 is formed of metal, and even in the case where the protective pipe 223 receives an impact from the outside and the actuator 22 and the protective pipe 223 are conducted, the generation of an abnormal voltage in the protective pipe 223 is suppressed.

### Modification 6 of the Embodiment

In the embodiment, the optical fiber attaching portion 24a is configured of a quadrangular fitting hole and the signal line arranging portion 24b is configured of a through groove provided on the outer periphery of the holding portion 24 along the axial direction. However, the holding portion is not limited to the configuration.

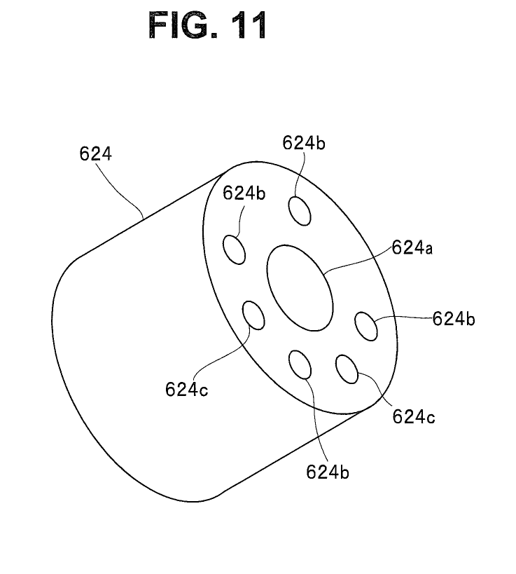

FIG. 11 is a perspective view illustrating a configuration of a holding portion 624 of a scanning endoscope, pertaining to the modification 6 of the embodiment of the present invention.

As illustrated in FIG. 11, an optical fiber attaching portion 624a may be a fitting hole in another shape, such as a circular fitting hole. In addition, a signal line arranging portion 624b may not be a groove on the outer periphery but may be a through-hole. Furthermore, a ground line arranging portion 624c may be a recessed portion provided on an end face.

Thus, in the holding portion 624, a groove is not formed on the outer periphery, the entire outer periphery of the holding portion 624 can be bonded with a portion of an inner periphery of the protective pipe 23 or 223, an area of a bonding part of the holding portion 624 and the protective pipe 23 or 223 is large, and electric resistance of the bonding part can be reduced.

The present invention is not limited to the above-described embodiment and can be variously changed and modified or the like without changing a subject matter of the present invention.

According to the present invention, the scanning endoscope, wherein the cylinder body at the distal end of the insertion portion is formed of metal, and which can suppress the generation of an abnormal voltage in the cylinder body even in the case where the actuator is brought into contact with the inner wall of the cylinder body due to an impact from the outside, can be provided.

The present application is filed claiming priority based on Japanese Patent Application No. 2016-053503 filed in Japan on Mar. 17, 2016, the disclosed contents of which are incorporated in the present description and the scope of claims by reference.

What is claimed is:

1. A scanning endoscope comprising:
an optical fiber for illumination including an incidence side to which illumination light enters and an irradiate side from which the illumination light is irradiated, and configured to irradiate an object from the irradiation side with the illumination light;
a holder configured to hold the optical fiber;
a plurality of drive elements arranged so as to make the optical fiber swing between the irradiation side and the holder, and configured to make the optical fiber swing according to a drive signal; and
a cylinder made of metal, and securing the optical fiber and the plurality of drive elements, wherein the cylinder is electrically connected to a ground potential.

2. The scanning endoscope according to claim 1,
wherein the holder is made of metal, and
wherein the cylinder is electrically connected to the holder.

3. The scanning endoscope according to claim 1, further comprising a ferrule configured to hold the optical fiber in a central axis thereof,
wherein the drive elements are arranged on an outer periphery of the ferrule, and
wherein the holder holds the ferrule.

4. The scanning endoscope according to claim 3,
wherein the ferrule is made of metal, and electrically connected to the holder.

5. The scanning endoscope according to claim 1,
wherein the holder comprises:
an optical fiber attaching portion for attaching the optical fiber;
a signal line arranging portion where a drive signal line configured to transmit the drive signal is arranged; and
a ground line arranging portion where a ground line configured to transmit the ground potential is arranged.

6. The scanning endoscope according to claim 5,
wherein the signal line arranging portion is insulated from the drive signal line,
the ground line arranging portion is electrically connected with the ground line; and
the holder is internally fitted to the cylinder such that at least a portion of an outer periphery of the holder and at least a portion of an inner wall of the cylinder are arranged to overlap each other, and is electrically connected with the cylinder.

7. The scanning endoscope according to claim 2, wherein the holder is fixed to the cylinder body with a conductive adhesive material.

8. The scanning endoscope according to claim 1,
wherein the cylinder is wound with a conductive material on at least a portion of an outer periphery thereof, and
the conductive material is connected to the ground potential.

9. The scanning endoscope according to claim 8,
wherein the conductive material is a metal sheet.

10. The scanning endoscope according to claim 1,
wherein the cylinder is made of metal integrated with the holder.

11. The scanning endoscope according to claim 1, comprising
a casing tube attached to the cylinder,
wherein the casing tube is including a metal mesh tube, and
the cylinder is connected to the metal mesh tube.
